(19) United States
(12) Patent Application Publication
(10) Pub. No.: US 2026/0219277 A1
(43) Pub. Date: Jul. 30, 2026

(54) NOVEL FLUORESCENT COMPOUND, AND COMPOSITION FOR DETECTING GINGIPAIN, COMPOSITION FOR DIAGNOSING PORPHYROMONAS GINGIVALIS INFECTION, OR ANTIBACTERIAL COMPOSITION AGAINST PORPHYROMONAS GINGIVALIS USING SAME

(71) Applicants: KOREA INSTITUTE OF SCIENCE AND TECHNOLOGY, Seoul (KR); SEOUL NATIONAL UNIVERSITY R&DB FOUNDATION, Seoul (KR)

(72) Inventors: Ae Nim PAE, Seoul (KR); Yun Kyung KIM, Seoul (KR); Sungsu LIM, Seoul (KR); Aizhan ABDILDINOVA, Seoul (KR); Lizaveta GOTINA, Seoul (KR); Hira AZIZ, Seoul (KR); Yu Jeong CHOI, Seoul (KR); Hyun Jeong KIM, Seoul (KR)

(73) Assignees: KOREA INSTITUTE OF SCIENCE AND TECHNOLOGY, Seoul (KR); SEOUL NATIONAL UNIVERSITY R&DB FOUNDATION, Seoul (KR)

(21) Appl. No.: 19/158,265

(22) PCT Filed: Jun. 19, 2024
(86) PCT No.: PCT/KR2024/008454

§ 371 Date: Aug. 20, 2025

(30) Foreign Application Priority Data
Jun. 19, 2023 (KR) 10-2023-0078048

(51) Int. Cl. G01N 33/58; A61K 31/41; A61P 31/04; C07D 257/04; G01N 33/573

(52) U.S. Cl. CPC G01N 33/582; A61K 31/41; A61P 31/04; C07D 257/04; G01N 33/573; G01N 2333/952; G01N 2800/18

(57) ABSTRACT

The present invention relates to a fluorescent compound represented by Chemical Formula 1 or a pharmaceutically acceptable salt thereof; and a composition for detecting gingipain, composition for diagnosing Porphyromonas gingivalis (P. gingivalis) infection or antibacterial composition against P. gingivalis using the same:

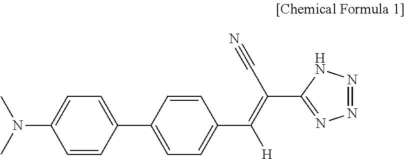 [SMILES: CN(C)c1ccc(-c2ccc(/C=C(\C#N)c3nnn[nH]3)cc2)cc1]

### TECHNICAL FIELD

The present invention relates to a novel fluorescent compound, and a composition for detecting gingipain, composition for diagnosing Porphyromonas gingivalis infection, or antibacterial composition against Porphyromonas gingivalis using same.

### BACKGROUND ART

Gingivalis or Porphyromonas gingivalis (P. gingivalis) is a key causative organism of gum disease and has been pointed out as a main cause of various systemic diseases, including cardiovascular disease. Gingipain, which is a proteolytic enzyme specifically secreted by P. gingivalis, not only decomposes the collagen that forms gum tissue but also disrupts vascular epithelial cells, allowing P. gingivalis to pass through the vascular barrier and invade various organs, causing inflammatory responses. P. gingivalis was reported to not only cause periodontitis/gingivitis, which is a major cause of tooth loss, but also cause cardiovascular, respiratory, musculoskeletal, and reproductive system-related abnormalities, leading to conditions such as atherosclerosis, diabetes, rheumatoid arthritis, and adverse pregnancy outcomes (APOs). In addition, recent studies have reported that P. gingivalis can pass through the blood brain barrier (BBB) and cause chronic inflammation in the brain, leading to brain diseases such as Parkinson's disease and Alzheimer's disease. In fact, it has been shown that gingipain is detected in more than 90% of the brain tissue of people who died from Alzheimer's disease.

Gingipain is a cysteine proteinase produced by P. gingivalis, and the nucleophilic cysteine thiol in the enzyme active site plays an important role in protein hydrolysis. There are two types of gingipain: arginine gingipains (RgpA and RgpB), which induce arginine-specific cleavage, and lysine gingipain (Kpg), which induces lysine-specific cleavage. Particularly, RgpA and Kgp are expressed as inactive precursors including a hemagglutinin-adhesion domain. Gingipain, activated by cleaving the hemagglutinin-adhesion domain, is transported and docked on the bacterial outer membrane and released into the extracellular environment. Here, an outer membrane vesicle (OMV) plays an important role in gingipain secretion and activity. The OMV is a structure similar to a spherical bilayer membrane having a diameter of about 50 to 250 nm, and is continuously released from the cell surface without the loss of the membrane during the growth of gram-negative bacteria. Activated gingipain is responsible for 85% of the extracellular proteolytic activity of P. gingivalis and essential for the survival of P. gingivalis by acquiring iron and nutrients.

To date, methods used to diagnose P. gingivalis infection in biological samples comprise (i) diagnosis of gingipain gene expression, (ii) gingipain-specific antibody-based diagnosis, and (iii) diagnosis of proteolytic activity of gingipain, but these methods can only confirm the presence or absence of P. gingivalis infection based on the activity of gingipain.

Therefore, when a fluorescent molecule diagnosis method that can easily detect or inhibit P. gingivalis and gingipain secreted by P. gingivalis is established, it is expected that not only periodontitis/gingivitis but also systemic inflammatory responses caused by P. gingivalis/gingipain can be diagnosed early, and the progression of the disease(s) can be prevented or treated.

### DISCLOSURE

### Technical Problem

The present invention is directed to providing a fluorescent compound represented by Chemical Formula 1 below or a pharmaceutically acceptable salt thereof for detecting gingipain, diagnosing Porphyromonas gingivalis (P. gingivalis) infection, or having an antibacterial action against P. gingivalis:

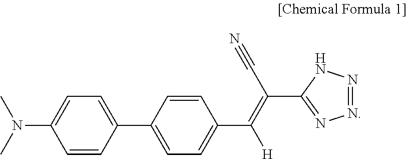 [SMILES: CN(C)c1ccc(-c2ccc(/C=C(\C#N)c3nnn[nH]3)cc2)cc1]

However, technical problems to be solved in the present invention are not limited to the above-described problems, and other problems which are not described herein will be fully understood by those of ordinary skill in the art from the following descriptions.

### Technical Solution

The present invention provides a fluorescent compound represented by Chemical Formula 1 below, or a pharmaceutically acceptable salt thereof:

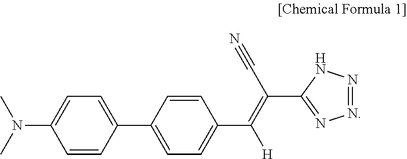 [SMILES: CN(C)c1ccc(-c2ccc(/C=C(\C#N)c3nnn[nH]3)cc2)cc1]

The fluorescent compound may be prepared by a reaction between Chemical Formula 1-1 below, malononitrile, and sodium azide (NaN₃):

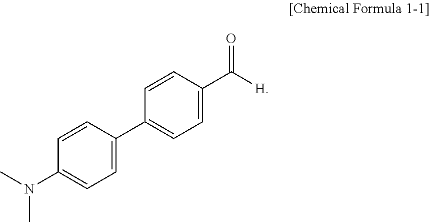

The reaction may be performed at 50 to 90° C. for 10 to 50 hours.

In one embodiment of the present invention, a fluorescent probe, which comprises the fluorescent compound or a pharmaceutically acceptable salt thereof, is provided.

The fluorescent compound or pharmaceutically acceptable salt thereof specifically binds to gingipain, but may not specifically bind to calpain or trypsin.

The gingipain may be secreted from P. gingivalis.

The gingipain may be arginine gingipain or lysine gingipain.

In another embodiment of the present invention, a composition for detecting gingipain, which comprises the fluorescent compound or a pharmaceutically acceptable salt thereof, is provided.

In still another embodiment of the present invention, a composition for diagnosing P. gingivalis infection, which comprises the fluorescent compound or a pharmaceutically acceptable salt thereof, is provided.

In yet another embodiment of the present invention, a method of visualizing gingipain, which comprises (a) treating a sample with the fluorescent compound or a pharmaceutically acceptable salt thereof; (b) generating fluorescence after the fluorescent compound or a pharmaceutically acceptable salt thereof binds to gingipain in the sample; and (c) observing the generated fluorescence, is provided.

The wavelength of the fluorescence may range from 450 to 700 nm.

In yet another embodiment of the present invention, an antibacterial composition against P. gingivalis, which comprises the fluorescent compound or a pharmaceutically acceptable salt thereof as an active ingredient, is provided.

In yet another embodiment of the present invention, a pharmaceutical composition for preventing or treating a P. gingivalis-infected disease, which comprises the fluorescent compound or a pharmaceutically acceptable salt thereof as an active ingredient, is provided.

The disease may be one or more selected from the group consisting of an inflammatory disease such as periodontitis or gingivitis; premature birth; a low birth weight; diabetes; rheumatoid arthritis; chronic lung disease; stroke; atherosclerosis; cardiovascular disease; heart disease; erectile dysfunction; and a neurodegenerative disease such as dementia or Parkinson's disease.

In yet another embodiment of the present invention, a food composition for preventing or improving a P. gingivalis-infected disease, which comprises the fluorescent compound or a pharmaceutically acceptable salt thereof as an active ingredient, is provided.

### Advantageous Effects

A novel fluorescent compound according to the present invention, specifically, a biphenyl-based compound or a pharmaceutically acceptable salt thereof can specifically bind to and inhibit gingipain, making it possible to detect gingipain, and selectively label and inhibit P. gingivalis, making it possible to diagnose P. gingivalis infection and also to have an antibacterial action against P. gingivalis.

### DESCRIPTION OF DRAWINGS

FIGS. 1A to 1F show the selection of (thiol-reactive) fluorescent compound DTC630 specifically binding to a cysteine residue in the gingipain active site.

FIGS. 2A to 2C show the Porphyromonas gingivalis (P. gingivalis) cell medium (P.g. C.M.)-specific fluorescence reaction and P. gingivalis cell-specific fluorescence reaction of the fluorescent compound DTC630.

FIGS. 3A to 3D show the analysis of the selectivity of the fluorescent compound DTC630 for gingipains (Kgp and Rgp) secreted from P. gingivalis.

FIGS. 4A to 4C show the analysis of the interaction between the fluorescent compound DTC630 and gingipains (Kgp and Rgp) secreted from P. gingivalis.

FIGS. 5A and 5B show the gingipain inhibitory effect of the fluorescent compound DTC630.

FIGS. 6A to 6E show the P. gingivalis inhibitory effect of the fluorescent compound DTC630 in HUVEC and HT29 cells.

FIGS. 7A and 7B show P. gingivalis-selective labeling of the fluorescent compound DTC630 in a tooth of a periodontitis patient.

### MODES OF THE INVENTION

In order to overcome the limitations mentioned above, the present inventors synthesized a biphenyl-based compound as novel fluorescent compound DTC630, and confirmed that, when specifically binding to and inhibiting gingipain, it can detect gingipain, when selectively labeling and inhibiting Porphyromonas gingivalis (P. gingivalis), it can diagnose P. gingivalis infection and have an antibacterial action against P. gingivalis, and thus completed the present invention.

Hereinafter, the present invention will be described in detail.

The present invention provides a fluorescent compound represented by Chemical Formula 1 below (or fluorescent compound DTC630), or a pharmaceutically acceptable salt thereof:

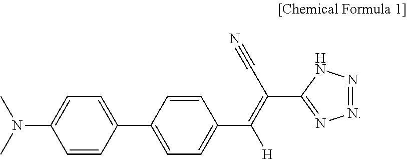 [SMILES: CN(C)c1ccc(-c2ccc(/C=C(\C#N)c3nnn[nH]3)cc2)cc1]

The fluorescent compound is a biphenyl-based compound, wherein biphenyl acts as the main fluorescence-generating moiety. Furthermore, the fluorescent compound has a tetrazole group and an acrylonitrile group at one end and an amine group at the other end. Here, when the tetrazole or acrylonitrile group is omitted, even with a similar structure, the compound may not exhibit gingipain and P. gingivalis-specific fluorescence and inhibitory reactions. In addition, when its molecular weight is too large, the gingipain and P. gingivalis-specific fluorescence and inhibitory reactions may not occur.

Specifically, the IUPAC name of the fluorescent compound is (E)-3-(4′-(dimethylamino)-[1,1′-biphenyl]-4-yl)-2-(1H-tetrazol-5-yl) acrylonitrile.

Meanwhile, the fluorescent compound may be prepared through a reaction between the following Chemical Formula 1-1, malononitrile, and sodium azide (NaN₃):

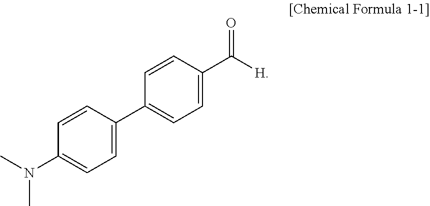

The reaction may be performed at 50 to 90° C. for 10 to 50 hours, and preferably at 70 to 80° C. for 20 to 30 hours, but the present invention is not limited thereto.

In addition, the present invention provides a fluorescent probe, which comprises the fluorescent compound or a pharmaceutically acceptable salt thereof.

The fluorescent compound or a pharmaceutically acceptable salt thereof may specifically bind to gingipain, and particularly, specifically bind to a cysteine residue in the active site of gingipain (thiol-reactive) and inhibit gingipain. However, the fluorescent compound or a pharmaceutically acceptable salt thereof may not specifically bind to calpain or trypsin.

The gingipain may be secreted from P. gingivalis. In addition, the gingipain may be arginine gingipain or lysine gingipain. Here, arginine gingipains (RgpA and RgpB) may induce arginine-specific cleavage, and lysine gingipain (Kpg) may induce lysine-specific cleavage.

In addition, the fluorescent compound or a pharmaceutically acceptable salt thereof may have fluorescence and inhibitory reactions specific to P. gingivalis cells or a P. gingivalis cell medium (P. gingivalis C.M.). However, the fluorescent compound or a pharmaceutically acceptable salt thereof may not have a fluorescence reaction specific to F. nucleatum cells or a F. nucleatum cell medium (F. nucleatum C.M.), A. actinomycetecomitans cells or A. actinomycetecomitans cell medium (A. actinomycetecomitans C.M.).

In addition, the present invention provides a composition for detecting gingipain, comprising the fluorescent compound or a pharmaceutically acceptable salt thereof; a use thereof in the composition for detecting gingipain; or a method of detecting gingipain using the same.

To detect gingipain, the fluorescent compound or a pharmaceutically acceptable salt thereof may specifically bind to gingipain, and particularly, a cysteine residue in the active site of gingipain and inhibit the activity of gingipain. This is a gingipain-specific reaction and does not specifically bind to a human enzyme such as calpain or trypsin, thereby reducing side effects.

In addition, the present invention provides a composition for diagnosing P. gingivalis infection, comprising the fluorescent compound or a pharmaceutically acceptable salt thereof; a use thereof in the composition for diagnosing P. gingivalis infection; or a method of diagnosing P. gingivalis infection using the same.

To diagnose P. gingivalis infection, the fluorescent compound or a pharmaceutically acceptable salt thereof may bind to gingipain present in P. gingivalis cells or a P. gingivalis cell medium to exhibit a fluorescence reaction and induce activity inhibition. This is a specific reaction to P. gingivalis expressing gingipain, and may not cause a fluorescence reaction specific to F. nucleatum cells or a F. nucleatum cell medium (F. nucleatum C.M.), or A. actinomycetecomitans cells or A. actinomycetecomitans cell medium (A. actinomycetecomitans C.M.).

In addition, the present invention provides a method of visualizing gingipain, which comprises (a) treating a sample with the fluorescent compound or a pharmaceutically acceptable salt thereof; (b) generating fluorescence after the fluorescent compound or a pharmaceutically acceptable salt thereof binds to gingipain in the sample; and (c) observing the generated fluorescence.

The wavelength of the fluorescence may range from 450 to 700 nm, and preferably, 500 to 600 nm.

In addition, the present invention provides an antibacterial composition against P. gingivalis, which comprises the fluorescent compound or a pharmaceutically acceptable salt thereof as an active ingredient.

Alternatively, the present invention provides a use of the fluorescent compound or a pharmaceutically acceptable salt thereof in an antibacterial composition against P. gingivalis.

Alternatively, the present invention provides an antibacterial method against P. gingivalis, which comprises administering the fluorescent compound or a pharmaceutically acceptable salt thereof to an individual. Here, “individual” refers to a subject in need of treatment for a disease, and more particularly, a mammal such as a human or a non-human primate, a mouse, a rat, a dog, a cat, a horse, or a cow.

The antibacterial composition is directed against P. gingivalis, and may be used in various ways, such as a pharmaceutical composition, a food or health functional food composition, and an oral composition.

In addition, the present invention provides a pharmaceutical composition for preventing or treating a P. gingivalis-infected disease, which comprises the fluorescent compound or a pharmaceutically acceptable salt thereof as an active ingredient.

Moreover, the present invention provides a food composition for preventing or treating a P. gingivalis-infected disease, which comprises the fluorescent compound or a pharmaceutically acceptable salt thereof as an active ingredient.

Alternatively, the present invention provides a use of the fluorescent compound or a pharmaceutically acceptable salt thereof in a composition for preventing, treating or alleviating a P. gingivalis-infected disease.

Alternatively, the present invention provides a method of preventing, treating or alleviating a P. gingivalis-infected disease, which comprises administering the fluorescent compound or a pharmaceutically acceptable salt thereof to an individual. Here, “individual” refers to a subject in need of treatment for a disease, and more particularly, a mammal such as a human or a non-human primate, a mouse, a rat, a dog, a cat, a horse, or a cow.

The disease may refer to a disease caused by P. gingivalis infection, which may be one or more selected from the group consisting of a system inflammatory disease such as periodontitis or gingivitis; premature birth; a low birth weight; diabetes; rheumatoid arthritis; chronic lung disease; stroke; atherosclerosis; cardiovascular disease; heart disease; erectile dysfunction; and a neurodegenerative disease, such as dementia or Parkinson's disease.

The pharmaceutical composition according to the present invention may be formulated and used in the form of an oral formulation such as a powder, granules, capsules, a suspension, an emulsion, a syrup, or an aerosol, a preparation for external use, a suppository, or a sterile injectable solution according to a conventional method, and may include an appropriate carrier, excipient or diluent conventionally used in the preparation of a pharmaceutical composition.

The carrier, excipient, or diluent may include various compounds or mixtures, such as lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

When formulating, commonly used diluents or excipients such as a filler, an extender, a binder, a wetting agent, a disintegrant, and a surfactant, may be used.

Solid formulations for oral administration may be prepared by mixing at least one excipient, for example, starch, calcium carbonate, sucrose, lactose, and gelatin, with the active ingredient. In addition, other than simple excipients, lubricants such as magnesium stearate and talc may also be used.

Liquid formulations for oral administration may include a suspension, a liquid for internal use, an emulsion, and a syrup, and include various excipients, such as a wetting agent, a sweetening agent, a flavoring agent, and a preservative, in addition to simple diluents such as water and liquid paraffin.

Formulations for parenteral administration may include sterilized aqueous solutions, non-aqueous solvents, suspensions, emulsions, freeze-dried formulations, or suppositories. As the non-aqueous solvent or suspension, propylene glycol, polyethylene glycol, a vegetable oil such as olive oil, or an injectable ester such as ethyl oleate may be used. As a suppository base, Witepsol, Tween 61, cacao butter, laurin butter, glycerol, or gelatin may be used.

A preferable dose of the pharmaceutical composition of the present invention may depend on a subject's condition and body weight, the severity of a disease, a drug type, an administration route and administration duration, and may be suitably selected by those of ordinary skill in the art. However, for a preferred effect, the pharmaceutical composition is preferably administered 0.0001 to 2,000 mg/kg, and more preferably, 0.001 to 2,000 mg/kg per day. The pharmaceutical composition may be administered once a day or in several divided doses. However, the above-mentioned dose does not limit the scope of the present invention.

The pharmaceutical composition according to the present invention may be administered to mammals such as a rat, a mouse, livestock, and a human through various routes. Any administration mode such as oral, rectal, intravenous, intramuscular, subcutaneous, intrauterine, intrathecal, or intracerebroventricular injection may be contemplated.

In the food or health functional food composition according to the present invention, when the active ingredient is used as an additive in health functional food, it may be added alone or may be used in combination with other food or food ingredients, and may be used appropriately according to a conventional method. The mixing amount of the active ingredient may be determined appropriately according to each purpose of use, such as prevention, health, or treatment.

Dosage forms of health functional food may include a powder, granules, pills, tablets, and capsules as well as general foods or beverages.

There is no particular limitation on the type of food. Examples of the food to which the above material can be added may include meat, sausage, bread, chocolate, candy, snacks, confectionaries, pizza, ramen, other noodles, gum, dairy products including ice cream, various types of soups, beverages, tea, drinks, alcoholic beverages and vitamin complexes, and in a general sense, can include all health functional foods.

Generally, in the production of foods or beverages, the active ingredient may be added at 15 parts by weight or less, and preferably, 10 parts by weight or less, with respect to 100 parts by weight of the raw materials. However, in the case of long-term intake for the purposes of health and hygiene or health control, the amount may be below the above range.

Among the health functional foods according to the present invention, beverages may contain various flavoring agents or natural carbohydrates as additional components, like ordinary beverages. The above-described natural carbohydrates may include monosaccharides such as glucose, fructose, etc.; disaccharides such as maltose, sucrose, etc.; and polysaccharides such as dextrin, cyclodextrin, etc., and sugar alcohols such as xylitol, sorbitol, erythritol, etc. As the sweeteners, natural sweeteners such as thaumatin and stevia extract and synthetic sweeteners such as saccharin and aspartame may be advantageously used. The proportion of the natural carbohydrates may be about 0.01 to 0.04 g, and preferably, about 0.02 to 0.03 g per 100 mL of the beverage according to the present invention.

In addition to the above, the health functional food composition according to the present invention may contain various nutrients, vitamins, electrolytes, flavoring agents, pectic acid and a salt thereof, alginic acid and a salt thereof, organic acids, protective colloidal thickening agents, pH adjustors, stabilizers, preservatives, glycerin, alcohols, or carbonizing agents used in carbonated beverages. In addition, the health functional food composition according to the present invention may contain pulp for producing natural fruit juice, fruit juice drinks, and vegetable drinks. Such components may be used independently or in combination. The proportion of the additive may generally be, but is not limited to, selected in the range of 0.01 to 0.1 parts by weight with respect to 100 parts by weight of the health functional food composition according to the present invention.

In the oral composition according to the present invention, the oral composition may be formulated in the form of a powder, a granule, a tablet, a capsule, a cream, a transparent gel, or a semi-transparent gel. That is, the oral composition is characterized in that it is not formulated in a liquid form such as a solution, a suspension, or an emulsion, and has the advantage of minimizing the side effects of existing oil pulling. For the formulation, one or more selected from the group consisting of an appropriate abrasive, humectant, binder, foaming agent, sweetening agent, preservative, medicinal agent, flavoring agent, acidity regulator and bleaching agent, which are conventionally used in the preparation of an oral composition may be further included. Here, the additive is preferably included at 0.01 to 10 parts by weight with respect to 100 parts by weight of the oral composition, but the present invention is not limited thereto.

The abrasive may be one or more selected from the group consisting of calcium monohydrogen phosphate, precipitated silica, calcium carbonate, hydrous alumina, kaolin, and sodium bicarbonate, and preferably calcium monohydrogen phosphate or precipitated silica.

The humectant may be one or more selected from the group consisting of glycerin, sorbitol, an amorphous sorbitol solution, propylene glycol, polyethylene glycol, and xylitol, and preferably, sorbitol.

The binder may be one or more selected from the group consisting of sodium carboxymethyl cellulose, carrageenan, xanthan gum, hydroxyethyl cellulose, hydroxypropyl methyl cellulose, hydroxypropyl cellulose, guar gum, gellan gum, carbomer, pectin, polyvinylpyrrolidone, carboxyvinyl polymer, sodium alginate, and laponite, and preferably, carboxymethyl cellulose.

The foaming agent may be one or more selected from the group consisting of anionic surfactants such as sodium lauryl sulfate and sodium lauryl sarcosinate, non-ionic surfactants such as sorbitan fatty acid ester, polyoxyethylene hydrogenated castor oil, and a polyoxyethylene-polyoxypropylene copolymer, and amphoteric surfactants such as cocoaidopropyl betaine, and preferably, sodium lauryl sulfate.

The sweetening agent may be one or more selected from the group consisting of sodium saccharin, sucralose, maltitol, aspartame, erythritol, and licorice acid, and preferably, sodium saccharin.

The preservative may be one or more selected from the group consisting of paraoxybenzoic acid ester and sodium benzoate, and preferably, paraoxybenzoic acid ester. The medicinal agent may be one or more selected from the group consisting of sodium fluoride, sodium fluorophosphate, stannous fluoride, amine fluoride, chlorhexidine, aminocaproic acid, dipotassium glycyrrhizinate, tranexamic acid, allotoin, caproic acid, polyphosphates, enzymes, and herbal extracts, and preferably, sodium fluoride.

The flavoring agent may be used by mixing suitable amounts of natural flavoring agents such as peppermint oil and spearmint oil, and synthetic flavoring agents such as menthol and carvone, and preferably, mixing a suitable amount of anise oil with the flavoring agents mixed in a certain ratio.

As mentioned above, the novel fluorescent compound according to the present invention, particularly, the biphenyl-based compound or pharmaceutically acceptable salt thereof may specifically bind to gingipain to detect gingipain, diagnose P. gingivalis infection by selectively labeling and inhibiting P. gingivalis, and exhibit an antibacterial action against P. gingivalis.

Hereinafter, preferred examples are presented to allow the present invention to be better understood. However, the following examples are merely provided to more easily understand the present invention, and the content of the present invention is not limited by the following examples.

### EXAMPLES

### Example 1: Synthesis of [(E)-3-(4′-(dimethylamino)-[1,1′-biphenyl]-4-yl)-2-(1H-tetrazol-5-yl) acrylonitrile] (fluorescent compound DTC630, Compound (4))

Fluorescent compound DTC630 (Compound (4)) was synthesized with reference to Reaction Scheme 1 below.

Specifically, after adding 4-bromobenzaldehyde (2) (0.412 g, 2.23 mmol) to a 4-(dimethylamino)phenylboric acid pinacol ester (1) (0.5 g, 2.02 mmol) solution in 1,4-dioxane (15 mL), K₃PO₄ (1.28 g, 6.06 mmol) and Pd(PPh₃)₄ (0.117 g, 0.101 mmol) were added. The reaction mixture was refluxed overnight. After completion, the reaction mixture was cooled to room temperature and poured into water. The formed precipitate was filtered, washed with water and then ether, and dried under vacuum, thereby obtaining Compound (3) (0.335 g, 73.6%): ¹H NMR (400 MHz, [D6] DMSO, 25° C., TMS): δ=9.98 (s, 1H), 7.91 (d, J=8.4 Hz, 2H), 7.84 (d, J=8.4 Hz, 2H), 7.66 (d, J=8.9 Hz, 2H), 2.97 ppm (s, 6H). ¹³C NMR (101 MHz, [D6] DMSO, 25° C., TMS): δ=192.83, 151.11, 146.57, 134.19, 130.68, 128.18, 126.08, 125.97, 112.96, 40.34 ppm. LC-MS (ESI, 60 eV) m/z=226.00 [M+H]⁺ HPLC (95.17%).

Afterward, after adding malononitrile (57 mg, 0.86 mmol) to a solution of Intermediate (3) (150 mg, 0.66 mmol) in EtOH (10 mL), NaN₃ (56 mg, 0.86 mmol) was added. The reaction mixture was refluxed for 24 hours until the aldehyde was completely converted. After completion, the reaction mixture was cooled to room temperature and concentrated under reduced pressure. The formed precipitate was washed with water, filtered, recrystallized with ethanol, and dried under vacuum, thereby obtaining Compound (4) (155 mg, 74%): ¹H NMR (400 MHz, [D4] MeOH, 25° C., TMS): δ=8.14 (s, 1H), 8.03 (d, J=8.4 Hz, 2H), 7.75 (d, J=8.5 Hz, 2H), 7.63 (d, J=8.9 Hz, 2H), 6.87 (d, J=8.9 Hz, 2H), 3.02 ppm (s, 6H). ¹³C NMR (101 MHz, [D4] MeOH, 25° C., TMS): δ=158.20, 150.79, 143.77, 143.58, 130.85, 129.86, 127.25, 127.18, 125.62, 116.75, 112.59, 99.09, 39.26 ppm. LC-MS (ESI, 60 eV) m/z=317.00 [M+H]⁺ HRMS (ESI) m/z calcd for C₁₈H₁₇N₆⁺: 317.1508 [M+H]⁺; found: 317.1508. HPLC (99.06%).

### Comparative Example 1: Synthesis of [2-((4′-(dimethylamino)-[1,1′-biphenyl]-4-yl)methylene)malononitrile] (fluorescent compound DTC2352, Compound (5))

Fluorescent compound DTC2352 (Compound (5)) was synthesized with reference to Reaction Scheme 1 below.

Specifically, Compound (3) (0.335 g, 73.6%) was obtained in the same manner as in Example 1. Afterward, after adding malononitrile (32 mg, 0.48 mmol) to a solution of Intermediate (3) (100 mg, 0.44 mmol) in EtOH (6 mL), piperidine (44 μL, 0.44 mmol) was added. The reaction mixture was stirred at room temperature for 3 hours until Intermediate (3) was completely converted. After completion, the reaction mixture was poured into ice water and filtered. The crude mixture was purified via flash column chromatography (EA:Hex, 1:4, v/v) to obtain Compound (5) (45 mg, 37%): ¹H NMR (400 MHz, [D6] DMSO, 25° C., TMS): δ=8.46 (s, 1H), 7.99 (d, J=8.3 Hz, 2H), 7.90 (d, J=8.3 Hz, 2H), 7.72 (d, J=8.7 Hz, 2H), 6.82 (d, J=8.7 Hz, 2H), 2.99 ppm (s, 6H). ¹³C NMR (101 MHz, [D6] DMSO, 25° C., TMS): δ=161.01, 151.40, 146.56, 132.05, 129.06, 128.28, 126.08, 125.21, 115.23, 114.33, 112.90, 78.89, 40.28 ppm. LC-MS (ESI, 60 eV) m/z=274.00 [M+H]⁺ HRMS (ESI) m/z calcd for C₁₈H₁₆N₃⁺: 274.1338 [M+H]⁺; found: 274.1336. HPLC (98.34%).

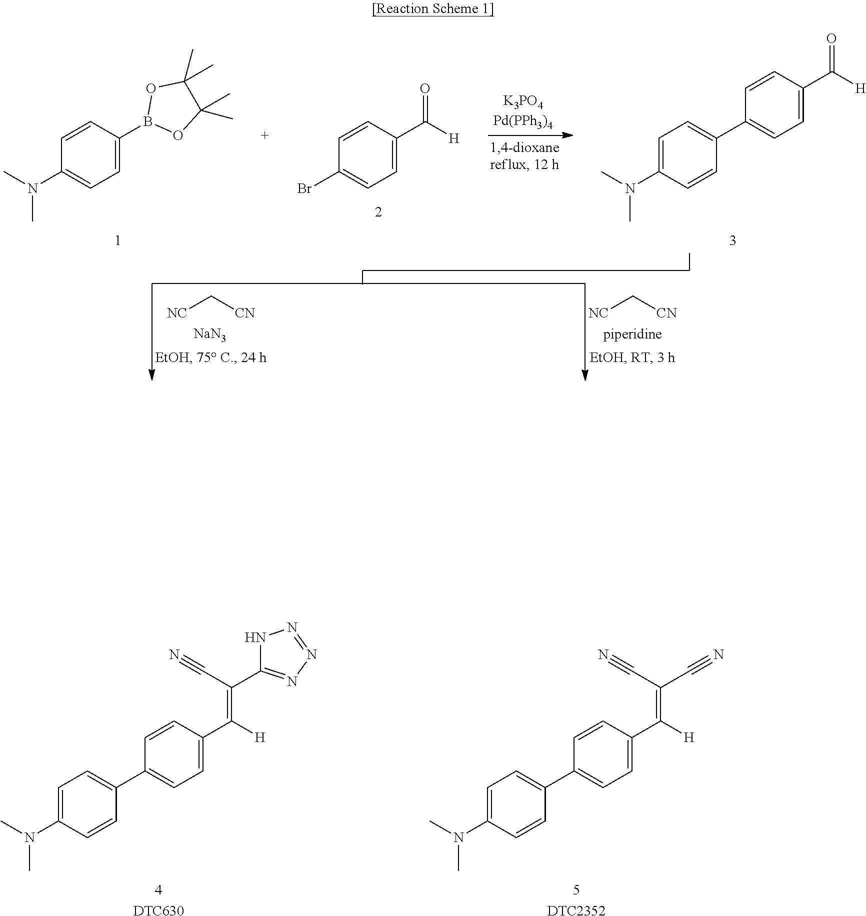

### Experimental Example 1: Selection of Fluorescent Compound DTC630

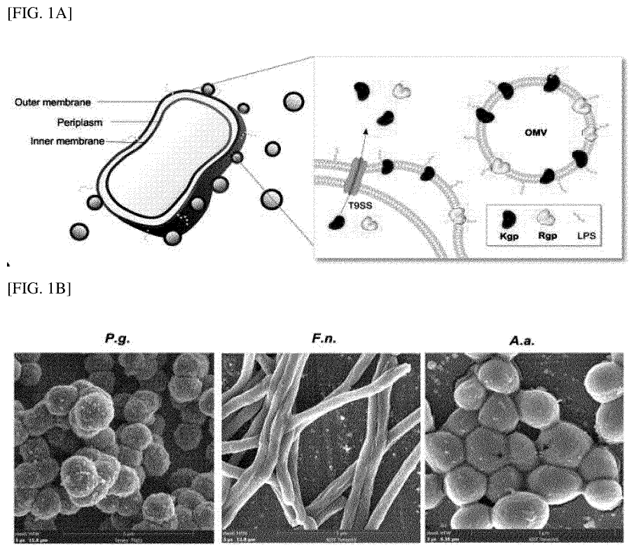

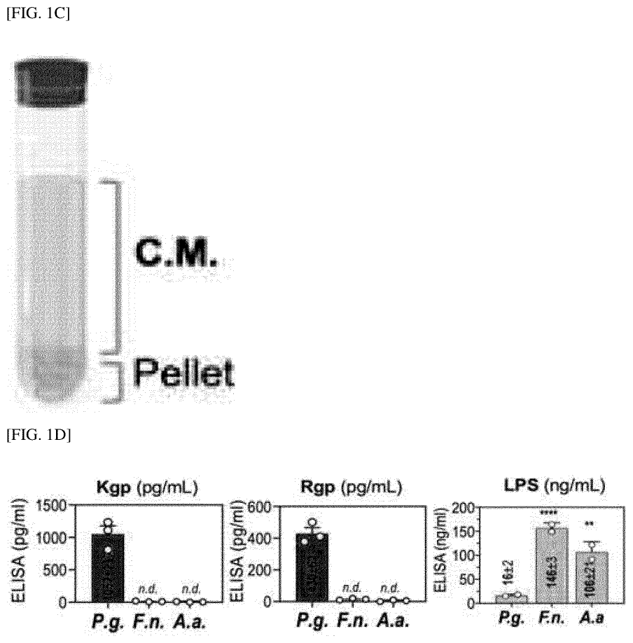

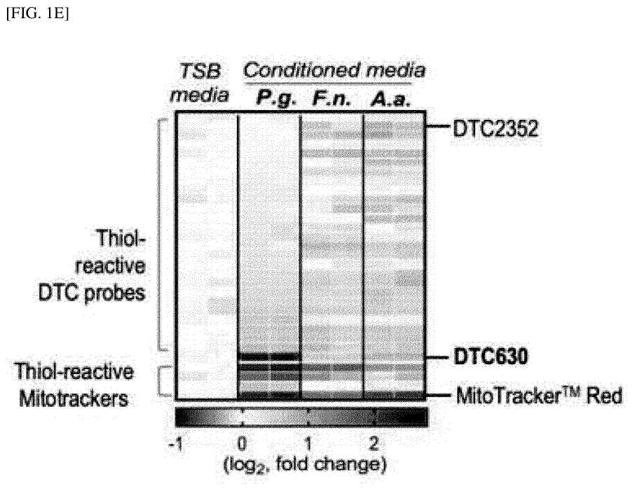

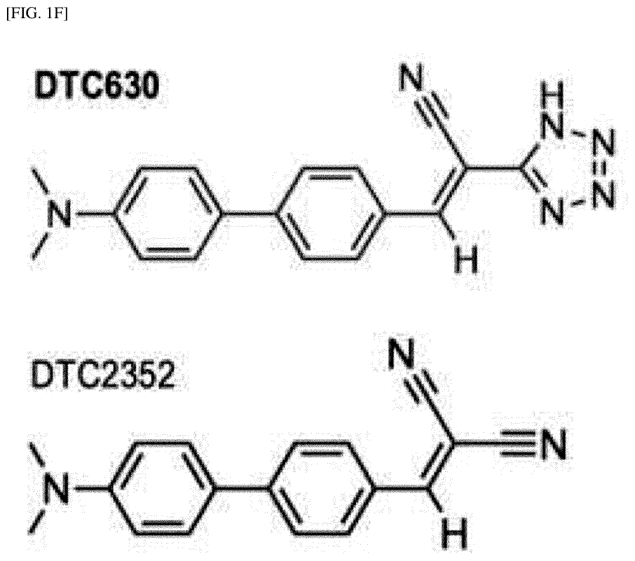

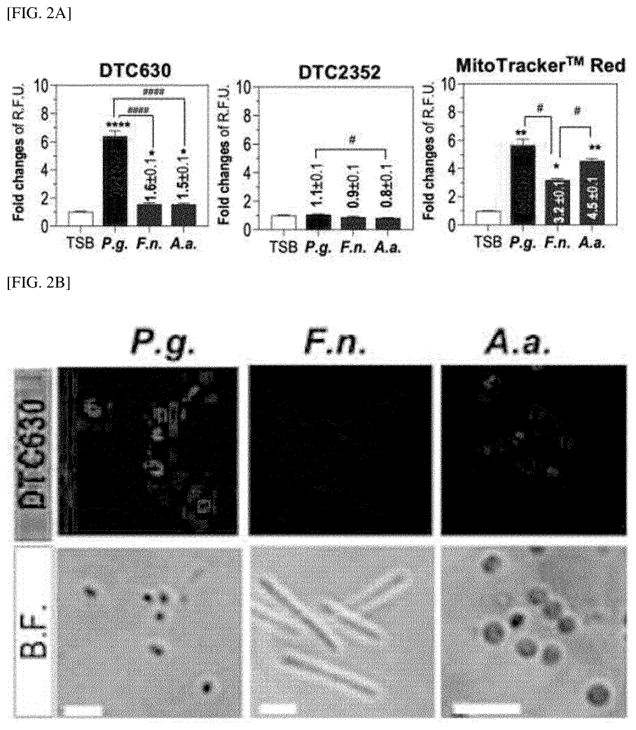

(Thiol reactive) fluorescent compound DTC630 specifically binding to a cysteine residue in the active site of gingipain was selected (FIG. 1).

Specifically, P. gingivalis is known to secrete gingipain (Kgp, Rgp) and outer membrane vesicles (OMVs) (FIG. 1A). Causative bacteria of periodontitis, i.e., P. gingivalis, F. nucleatum, and A. actinomycetecomitans, were observed using a scanning electron microscope (SEM) (FIG. 1B). Afterward, the conditioned media (C.M.) were isolated (FIG. 1C).

As a result of quantifying gingipains (Kgp and Rgp) and an essential cell wall component (LPS) based on ELISA, the P. gingivalis conditioned medium (P.g. C.M.) showed analysis results of Kgp 1052±217 pg/mL, Rgp 430±62 pg/mL, and LPS 16±2 pg/mL. Meanwhile, the F. nucleatum conditioned medium (F.n. C.M.) showed analysis results of LPS 146±3 pg/mL, and the A. actinomycetecomitans conditioned medium (A.a C.M.) showed analysis results of LPS 106±21 pg/mL. That is, in the P. gingivalis conditioned medium (P.g. C.M.), gingipains (Kgp and Rgp) were specifically detected, but the essential cell wall component (LPS) was confirmed to be detected to a relatively small extent (FIG. 1D).

Therefore, the present inventors screened 24 types of thiol-reactive fluorescent compound libraries using the conditioned media. Here, four types of thiol-reactive MitoTrackers were used as control materials. As a result of analyzing the heatmap based on the fluorescence magnification for TSB medium, among the thiol-reactive fluorescent compound libraries, it was confirmed that only fluorescent compound DTC630 has considerably high selectivity for the P. gingivalis conditioned medium (P.g. C.M.) (FIG. 1E). That is, it was confirmed that fluorescent compound DTC630 exhibits a P. gingivalis-selective fluorescence reaction, whereas fluorescent compound DTC2352 having a similar structure with the above compound does not exhibit a P. gingivalis-selective fluorescence reaction (FIG. 1F).

### Experimental Example 2: Fluorescence Reaction of Fluorescent Compound DTC630

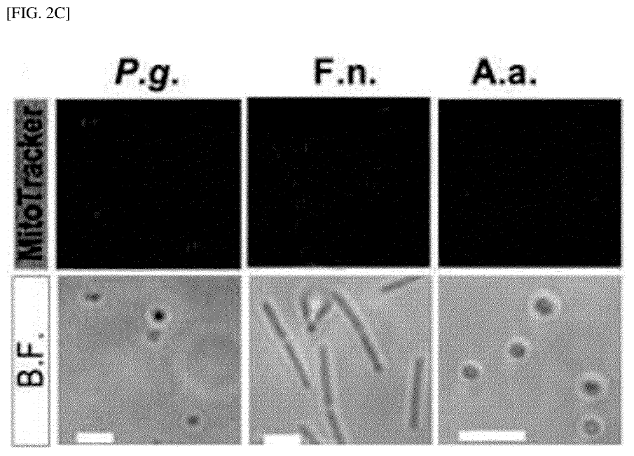

Fluorescent compound DTC630 exhibited a P. gingivalis conditioned medium (P.g. C.M.)-specific fluorescence reaction and a P. gingivalis cell-specific fluorescence reaction (FIG. 2).

Specifically, when fluorescent compound DTC630 was reacted in the conditioned media (Conditioned media, C.M.), compared to the basic TSB media for cell culture, a 6.2±0.2-fold increase in fluorescence was shown in the P. gingivalis conditioned medium (P.g. C.M.), a 1.6±0.1-fold increase in fluorescence was shown in the F. nucleatum conditioned medium (F.n. C.M.), and a 1.5±0.1-fold increase in fluorescence was shown in the A. actinomycetecomitans conditioned medium (A.a C.M.). By using MitoTracker™ Red among the MitoTackers, a 5.6±0.6-fold increase in fluorescence was shown in the P. gingivalis conditioned medium (P.g. C.M.), a 3.2±0.1-fold increase in fluorescence in the F. nucleatum conditioned medium (F.n. C.M.), and a 4.5±0.1-fold increase in fluorescence was shown in A. actinomycetecomitans conditioned medium (A.a C.M.). That is, only fluorescent compound DTC630 showed selectivity for the P. gingivalis conditioned medium (P.g. C.M.) (FIG. 2A). After culturing living P. gingivalis, F. nucleatum, and A. actinomycetecomitans with the fluorescent compound DTC630, and MitoTracker™ Red, cell-specific fluorescence reactions were compared. As a result, the fluorescent compound DTC630 showed selectivity by labeling only P. gingivalis (FIGS. 2B and 2C).

### Experimental Example 3: Selectivity of Fluorescent Compound DTC630 for Gingipains (Kgp and Rgp)

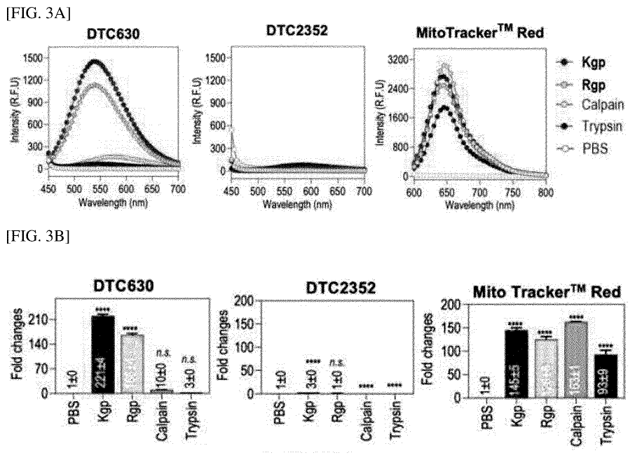

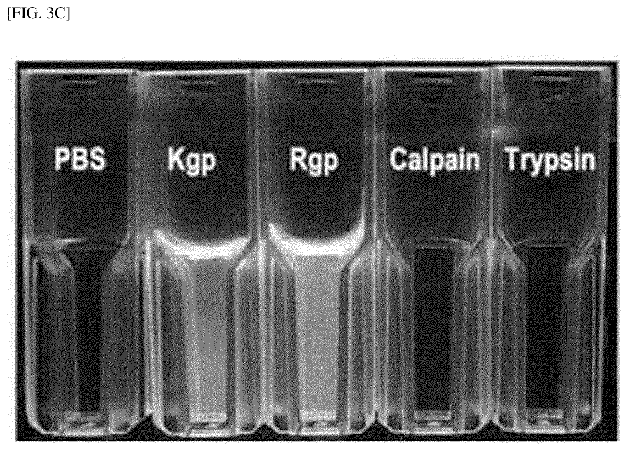

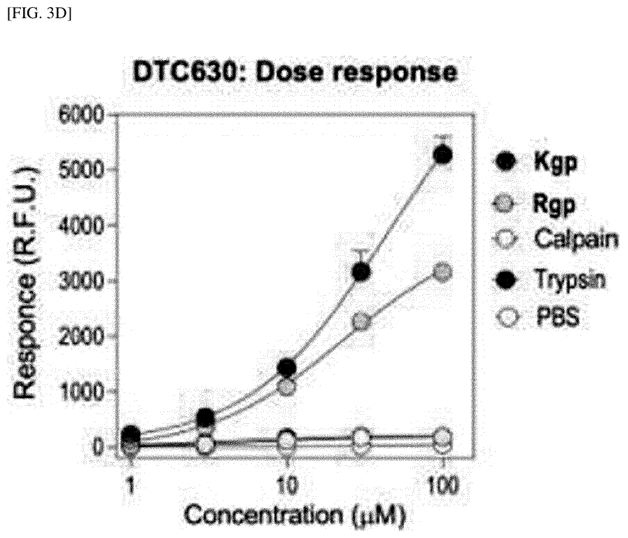

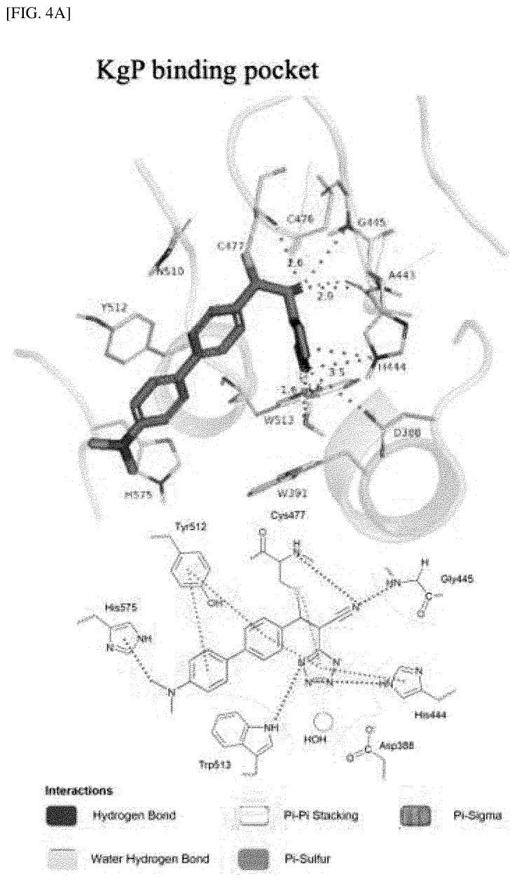

The selectivity of fluorescent compound DTC630 for gingipains (Kgp and Rgp) secreted from P. gingivalis was analyzed (FIG. 3).

Specifically, after the gingipains (Kgp and Rgp) secreted from P. gingivalis, calpain, which is a cysteine protease, and trypsin, which is a serine protease, were cultured with fluorescent compound DTC630 (10 μM), a fluorescence spectrum was measured (λₑₓ=425 nm, λₑₘ=450-700 nm).

As a result, fluorescent compound DTC630 selectively detected Kgp and Rgp to show a fluorescence increase compared to a PBS solution. Fluorescent compound DTC630 did not detect calpain and trypsin, so there was no increase in fluorescence. Meanwhile, the control, Compound DTC2325, did not detect any of Kgp, Rgp, calpain, and trypsin, so there was no increase in fluorescence. Mitotracker™ Red detected all of Kgp, Rgp, calpain, and trypsin, showing an increase in fluorescence compared to PBS (FIG. 3A).

In other words, fluorescent compound DTC630 showed significant fluorescence increases for Kgp (221±4-fold) and Rgp (166±4-fold) compared to the PBS solution due to selectivity for Kgp and Rgp, but did not exhibit selectivity for calpain and trypsin. The control, Compound DTC2325, showed a fluorescence increase for Kgp (3±0-fold) compared to PBS, but its basic R.F.U. value was insignificant. Mitotracker™MRed showed significant fluorescence increases for all of Kgp (145±5-fold), RgpA (126±6-fold), calpain (163±1-fold), and trypsin (93±9-fold), compared to PBS, without any particular selectivity (FIG. 3B).

Meanwhile, each of the reactants, PBS, Kgp, Rgp, calpain, and trypsin, which reacted with the fluorescent compound DTC630, was contained in a glass cuvette, and their fluorescence images were obtained with ultraviolet light (UV). Only the reactants Kgp and Rgp, for which fluorescent compound DTC630 exhibited selectivity, showed fluorescence (FIG. 3C).

In addition, the degree of fluorescence reaction of fluorescent compound DTC630 (1, 3, 10, 30, and 100 μM) was measured at each concentration for Kgp, Rgp, calpain, and trypsin (final concentration: 0.3 mg/mL). As a result, fluorescent compound DTC630 selectively labeled Kgp and Rgp and showed an increase in fluorescence by concentration (FIG. 3D).

### Experimental Example 4: Interaction of Fluorescent Compound DTC630 with Gingipains (Kgp and Rgp)

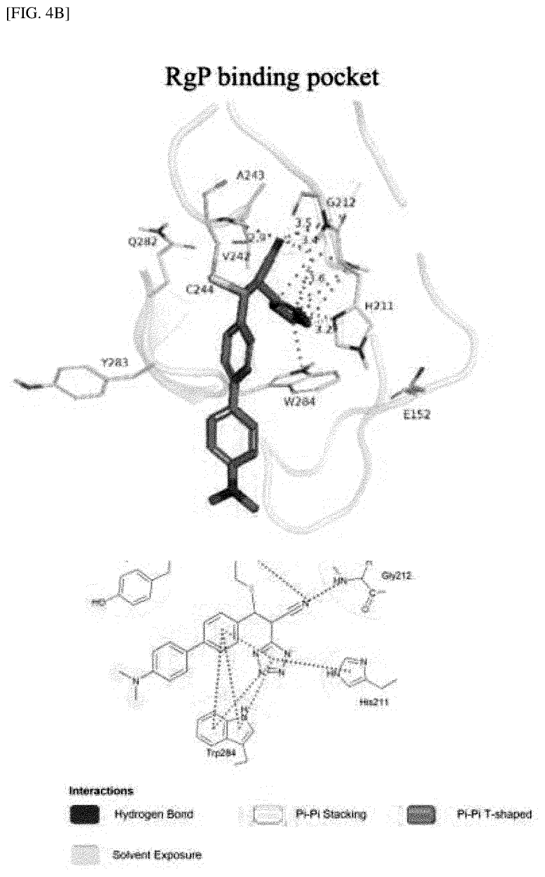

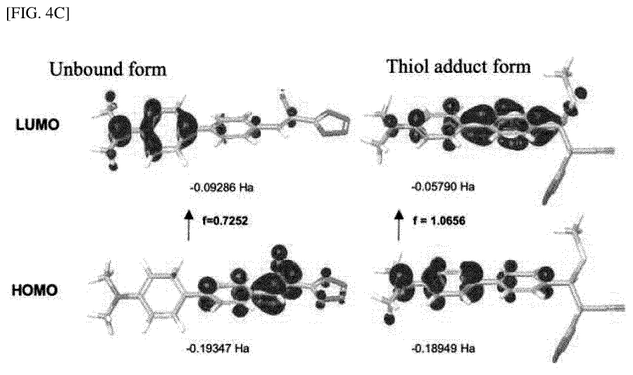

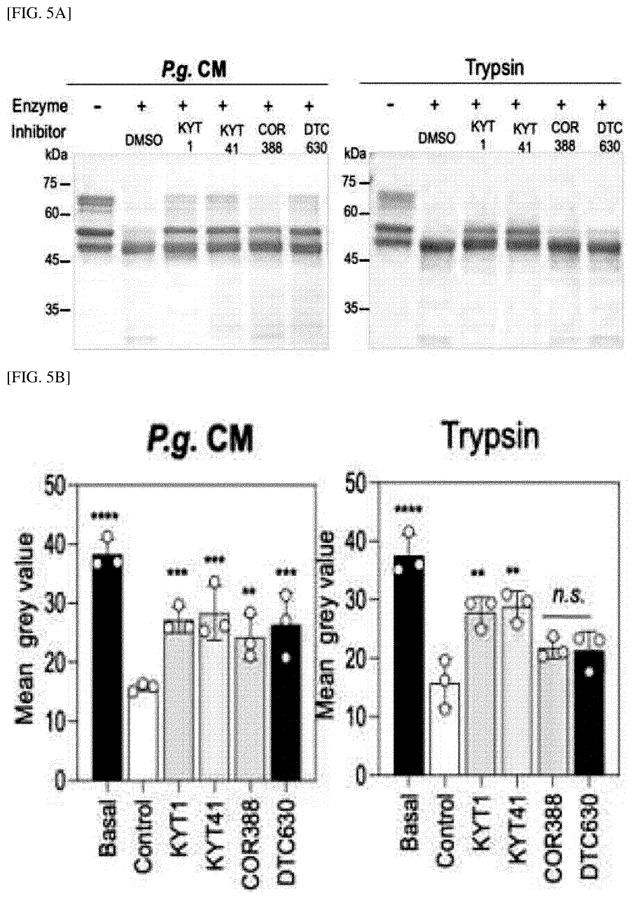

The interaction between fluorescent compound DTC630 and gingipains (Kgp and Rgp) secreted from P. gingivalis was analyzed (FIG. 4).

Specifically, in a Kgp-binding pocket (protein PDB ID: 619A) and an Rgp-binding pocket (protein PDB ID: 1CVR), a covalent docking binding mode of fluorescent compound DTC630 was shown (FIGS. 4A and 4B). The 3D images included key interacting residues in the form of thin rods and a polar contact map (magenta dotted lines with labeled distances (Å) between pairs of atom). Protein-ligand interactions are shown in a 2D interaction map. In Kgp, both the nitrile and tetrazole residues were located in the S1′ subsite (catalytic triad, Cys477, His444, and Asp388 subsite), and a 4-(dimethylamino) biphenyl group was located in the S3 (hydrophobic residue) subsite. Nitrile formed backbone H-bonds with Cys477 and Gly445, whereas tetrazole formed a direct H-bond and a π-π stacking interaction with His444, a water-mediated H-bond with Asp388, and an H-bond with Trp513. Biphenyl was well placed at Tyr512 to form a π-π stacking interaction, and dimethylamino had a hydrophobic interaction with His575. Since the available crystal structure for Rgp suggests a much shallower binding pocket in which Tyr283 and Glu152 are further away from the catalytic triad, the docking score of fluorescent compound DTC630 in RgP was predicted to be much weaker than that in Kgp (−2.533 vs. −4.061, respectively). In this case, fluorescent compound DTC630 may still bind in a “folded” form, but nitrile and tetrazole have moved closer to the S1 selectivity pocket. While nitrile maintained the H-bond network, tetrazole formed a π-π T-shaped interaction with His211 and π-π stacking with Trp284. Biphenyl π was stacked against Trp284 in a T-shaped conformation. Considering the overall shape of the ligand conformation and the abundance of hydrophobic π-π interactions in both binding pockets, the present inventors predicted that an aromatic group in the fluorescent compound DTC630 is more suppressed in the binding pocket which may favor more intensive fluorescence as shown for other protein-dye complexes.

The frontier molecular orbitals of fluorescent compound DTC630 and a corresponding thiol adduct, calculated at the B3LYP/6-311G(d,p) level in an aqueous solution are shown (FIG. 4C). To determine the fluorescence characteristics of the protein-bound and unbound forms of fluorescent compound DTC630, the present inventors performed TD-DFT calculations using a ligand docking pose in simplified thiol adduct form of KgP representing a binding state. The molecular orbital of the fluorescent compound DTC630 thiol adduct showed that a dimethylamine group was involved in charge transfer, and the main fluorescence-generating moiety was biphenyl. The higher LUMO energy of the thiol adduct was most associated with the destruction of a ligand-conjugated π-bond system compared to the unbound form. Oscillator intensity calculations indicated that the probability of the S0→S1 transition was higher in the fluorescent compound DTC630 thiol adduct compared to the ligand-unbound form.

### Experimental Example 5: Gingipain Inhibitory Effect of Fluorescent Compound DTC630

The gingipain inhibitory effect of fluorescent compound DTC630 is shown (FIG. 5). Specifically, to access the gingipain inhibitory effect of fluorescent compound DTC630, the present inventors cultured fibrinogen with a P. gingivalis conditioned medium (P.g. C.M.) in the presence or absence of 100 μM of fluorescent compound DTC630, the control inhibitor, KYT1 (Rgp inhibitor), KYT41 (Kgp/Rgp inhibitor), and COR388 (Kgp inhibitor). After 10 minutes of culture, the P. gingivalis conditioned medium (P.g. C.M.) was able to sufficiently degrade fibrinogen without any inhibitor (41.1±1.7%). It was observed that when the P. gingivalis conditioned medium (P.g. C.M.) was treated together with fluorescent compound DTC630 in a similar manner to the control inhibitor, fibrinogen degradation (68.8±13.8%) could be relieved (FIG. 5A).

In the presence or absence of 100 μM of KYT1, KYT41, COR388, and DTC630, SDS-PAGE gel analysis for the fibrinogen degradation performed using the P. gingivalis conditioned medium (P.g. C.M.) and trysin was shown (FIG. 5B). Here, quantification of fibrinogen degradation by ImageJ data was expressed as mean±SD from three independent experiments.

### Experimental Example 6: Gingivalis (P. gingivalis) Inhibitory Effect of Fluorescent Compound DTC630 in HUVEC and HT29 Cells

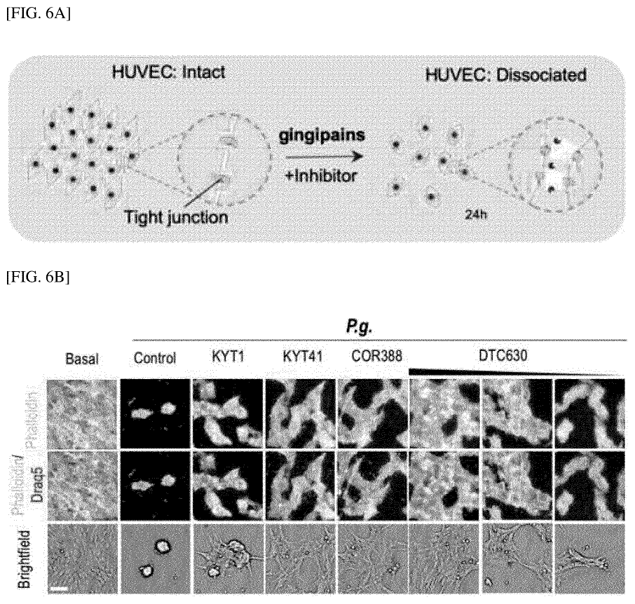

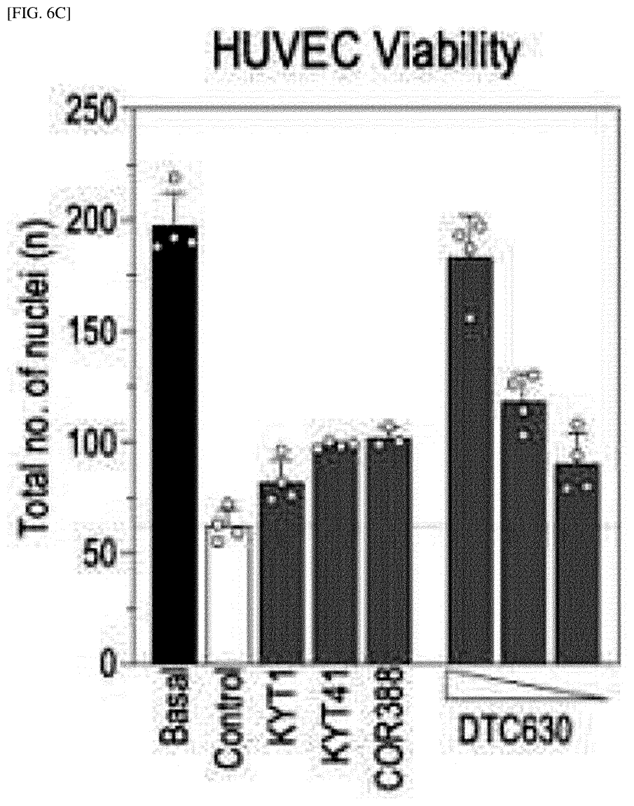

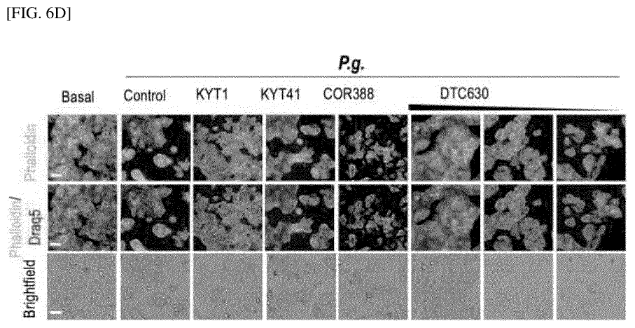

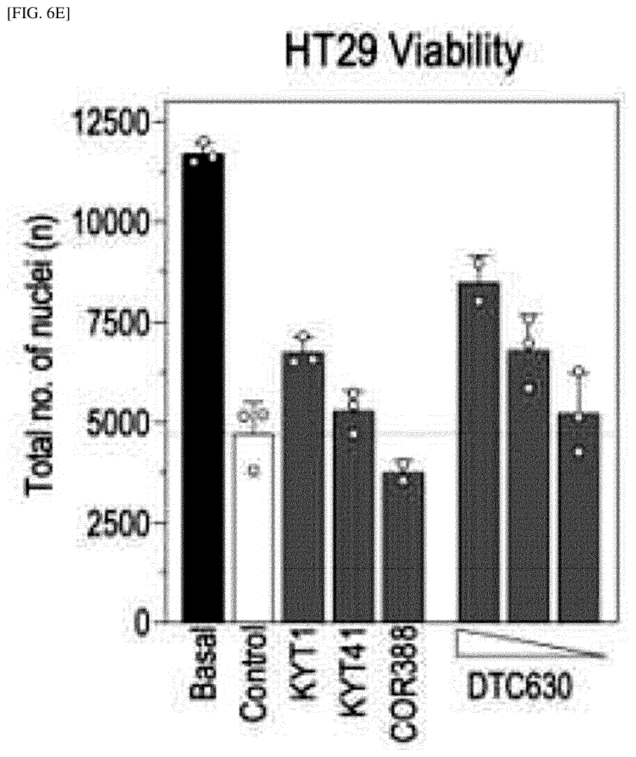

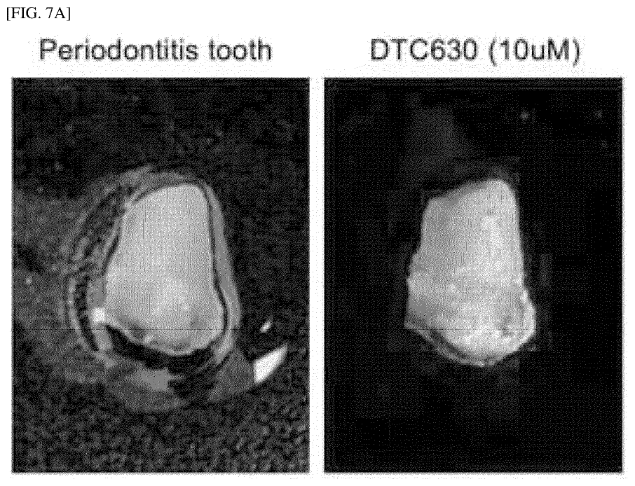

The P. gingivalis inhibitory effect of fluorescent compound DTC630 in HUVEC and HT29 cells is shown (FIG. 6).

First, it has been reported that P. gingivalis can successfully invade periodontal pockets in the oral cavity and then invade vascular endothelial cells and activate gingipain to cut blood vessels. Specifically, the experimental diagram for the P. gingivalis inhibitory effect of the fluorescent compound DTC630 in HUVEC cells is shown (FIG. 6A). The representative images of nuclear staining with Draq5 and cytoskeletal staining with phalloidin on the HUVEC cells treated with a P. gingivalis culture grown in the presence or absence of fluorescent compound DTC630, a control inhibitor, KYT1, KYT41, and COR388 are shown (FIG. 6B). Draq5 and phalloidin staining, indicating HUVEC viability and cell integrity, respectively, was quantified (FIG. 6C). Here, data was expressed as mean±SD based on four repetitions per treatment.

Subsequently, the representative images of the nuclear staining with Draq5 and cytoskeletal staining with phalloidin on HT29 cells treated with the P. gingivalis culture grown in the presence or absence of fluorescent compound DTC630, the control inhibitor, KYT1, KYT41, and COR388 are shown (FIG. 6D). Draq5 and phalloidin staining indicating HT29 viability and cell integrity, respectively, was quantified (FIG. 6E). Here, data was expressed as mean±SD based on four repetitions per treatment.

### Experimental Example 7: Gingivalis (P. gingivalis)-Selective Labeling of Fluorescent Compound DTC630 in Tooth of Periodontitis Patient

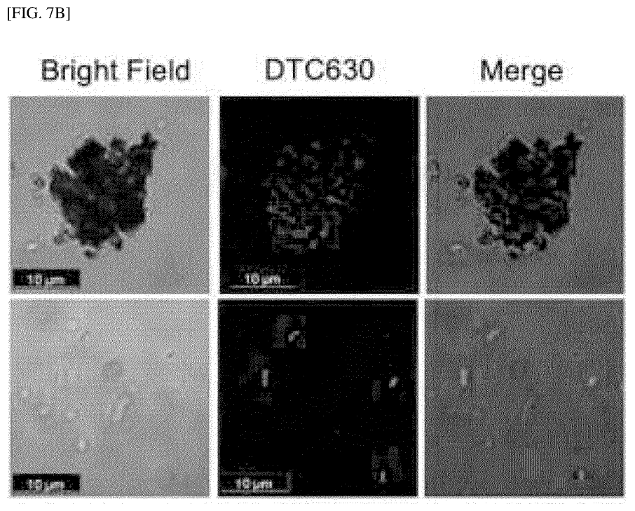

The selective labeling of P. gingivalis by fluorescent compound DTC630 in a tooth of a periodontitis patient is shown (FIG. 7).

Specifically, after culturing a tooth of a periodontitis patient and fluorescent compound DTC630 (10 μM), the fluorescence image of the tooth was observed. P. gingivalis present on the tooth surface was identified by fluorescence staining (FIG. 7A). An oral biofilm of periodontal disease-causing bacteria obtained by scraping the surface of the tooth of the periodontitis patient, was cultured with fluorescent compound DTC630 (10 μM), and then observed using bright field and fluorescence imaging. As a result, it was confirmed that fluorescent compound DTC630 selectively labels P. gingivalis present in the oral biofilm of the periodontal disease-causing bacteria (FIG. 7B).

It should be understood by those of ordinary skill in the art that the above description of the present invention is exemplary, and the example embodiments disclosed herein can be easily modified into other specific forms without changing the technical spirit or essential features of the present invention. Therefore, it should be interpreted that the example embodiments described above are exemplary in all aspects, and are not limitative.

1. A fluorescent compound represented by Chemical Formula 1 below or a pharmaceutically acceptable salt thereof:

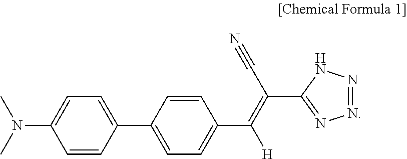 [SMILES: CN(C)c1ccc(-c2ccc(/C=C(\C#N)c3nnn[nH]3)cc2)cc1]

2. The fluorescent compound of claim 1, wherein the fluorescent compound is prepared by a reaction between Chemical Formula 1-1 below, malononitrile, and sodium azide (NaN₃):

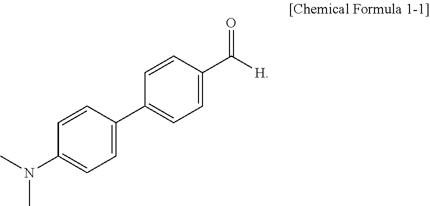

3. The fluorescent compound of claim 2, wherein the reaction is performed at 50 to 90° C. for 10 to 50 hours.

4. A fluorescent probe comprising the fluorescent compound or a pharmaceutically acceptable salt thereof according to claim 1.

5. The fluorescent probe of claim 4, wherein the fluorescent compound or pharmaceutically acceptable salt thereof specifically binds to gingipain, but does not specifically bind to calpain or trypsin.

6. The fluorescent probe of claim 5, wherein the gingipain is secreted from Porphyromonas gingivalis (P. gingivalis).

7. The fluorescent probe of claim 5, wherein the gingipain is arginine gingipain or lysine gingipain.

8. A method for detecting gingipain, using the fluorescent compound or pharmaceutically acceptable salt thereof of claim 1.

9. A method for diagnosing Porphyromonas gingivalis (P. gingivalis) infection, using the fluorescent compound or pharmaceutically acceptable salt thereof of claim 1.

10. A method of visualizing gingipain, comprising:
(a) treating a sample with the fluorescent compound or a pharmaceutically acceptable salt thereof;
(b) generating fluorescence after the fluorescent compound or a pharmaceutically acceptable salt thereof binds to gingipain in the sample; and
(c) observing the generated fluorescence.

11. The method of claim 10, wherein the wavelength of the fluorescence ranges from 450 to 700 nm.

12. An antibacterial method against Porphyromonas gingivalis (P. gingivalis), which comprises administering the fluorescent compound or pharmaceutically acceptable salt thereof of claim 1 to an individual.

13. A method for preventing or treating Porphyromonas gingivalis (P. gingivalis) infection, which comprises administering the fluorescent compound or pharmaceutically acceptable salt thereof of claim 1 to an individual.

14. The pharmaceutical method of claim 13, wherein the disease is one or more selected from the group consisting of an inflammatory disease such as periodontitis or gingivitis; premature birth; a low birth weight; diabetes; rheumatoid arthritis; chronic lung disease; stroke; atherosclerosis; cardiovascular disease; heart disease; erectile dysfunction; and a neurodegenerative disease, such as dementia or Parkinson's disease.

15. (canceled)
